SEQUENCE LISTING (7 sequences)

SEQ ID NO: 1 - protein, 9 aa, Influenza virus
GILGFVFTL

SEQ ID NO: 2 - protein, 9 aa, Influenza virus
NMLSTVLGV

SEQ ID NO: 3 - protein, 9 aa, Influenza virus
CVNGSCFTV

SEQ ID NO: 4 - protein, 9 aa, Influenza virus
SLENFRAYV

SEQ ID NO: 5 - protein, 10 aa, Influenza virus
IMDKNIILKA

SEQ ID NO: 6 - protein, 10 aa, Influenza virus
SLCPIRGWAI

SEQ ID NO: 7 - protein, 9 aa, Influenza virus
FMYSDFHFI

(19) United States
(12) Patent Application Publication
(10) Pub. No.: US 2011/0300168 A1
(43) Pub. Date: Dec. 8, 2011

(54) COMPOSITIONS AND METHODS FOR INDUCING AN IMMUNE RESPONSE AGAINST INFLUENZA ANTIGENS

(75) Inventors: Jonathan Schneck, Silver Spring, MD (US); Mathias Oelke, Baltimore, MD (US)

(73) Assignee: THE JOHNS HOPKINS UNIVERSITY, Baltimore, MD (US)

(21) Appl. No.: 13/099,514

(22) Filed: May 3, 2011

Related U.S. Application Data
(60) Provisional application No. 61/330,653, filed on May 3, 2010.

(51) Int. Cl. A61K 39/135; A61P 31/16

(52) U.S. Cl. 424/186.1

(57) ABSTRACT

This disclosure provides compositions and methods for inducing an immune response against influenza antigens, for example in elderly populations, as well as a sensitive artificial antigen presenting cell (aAPC) stimulation assay that can be used for expansion and analysis of multiple antigen specific T cell populations simultaneously.

This application claims the benefit of and incorporates by reference Ser. No. 61/330,653 filed on May 3, 2010.

Work described in this specification was funded by National Institutes of Health grants AI29575, CA108835, AI077097, and AI077056. The U.S. government therefore has certain rights in the invention.

This application incorporates by reference the contents of a 1.2 kb text file created on May 3, 2011 and named “sequencelisting.txt,” which is the sequence listing for this application.

Each patent, published patent application, and reference cited in this disclosure is expressly incorporated herein by reference in its entirety.

### FIELD OF THE INVENTION

Influenza infects millions of people worldwide every year (1). In the United States, it is estimated that more than 30,000 people die each year as a result of infection, with over 90% of deaths in individuals over age 65 (2, 3). Influenza pandemics can lead to much higher rates of mortality. Pandemics occur when a new strain of influenza begins to circulate that has not been previously seen by a population and is due to the antigenic shift when several influenza viruses recombine and result in significant changes the hemagglutinin (HA) and neuraminidase (NA) glycoproteins expressed on the viral surface (4).

While the flu vaccine is approximately 80% protective against influenza infection in healthy adults under the age of 65 (5), it does not protect everyone and it may not be available or protective in a worldwide pandemic. Furthermore, the effectiveness of the vaccine drops in persons aged 65 and above to as low as 30% (6-9). This is due, in part, to the diminished immune response in the elderly (10-14). While antibodies protect against development of primary influenza infection, clearance of the infection is chiefly mediated through CD8⁺ T cells (15, 16). It has been shown that CD8⁺ T cells are protective against influenza infection and are critical for the clearance of influenza infection in animal models (16-21).

While there is a vast array of potential CD8⁺ T cell antigens to be recognized, many infections have an immunodominant epitope to which most, if not all, of the response is directed. In influenza, the HLA-A2 restricted immunodominant response is to the matrix protein peptide, M1₅₈₋₆₆, and has been well characterized (22-24). However, some viruses, such as Hepatitis B, Hepatitis C, and HIV, do not elicit an immunodominant response (25-27). Rather there is a diverse array of epitopes against which T cells are directed. Similarly, recent studies of influenza have also shown a wide array of epitopes, suggesting that infection with influenza A may induce a broader response than a single immunodominant epitope (22, 28-31). Based on those studies (31), an alternative definition has been proposed of the hierarchy of dominant and subdominant epitopes for human immune responses. This definition is based on the frequency and magnitude of responses, is distinctive from those that feature absolute immunogenicity and recognition of naturally processed antigen (32).

There is a need in the art for sensitive methods for probing the breadth and depth of the human CD8⁺ T cell repertoire against influenza and which can be used to develop vaccine compositions.

### BRIEF DESCRIPTION OF THE DRAWINGS

FIGS. 1A-B. Precursor frequencies of influenza-specific CD8⁺ T cells are detectable by ELISpot, but at much lower levels for the subdominant epitopes. Legend: CD8⁺ T cells were obtained from control donor PBMCs directly ex vivo and analyzed by ELISpot. FIG. 1A. Shows a representative example of ELISpot wells. T2 cells were pulsed with either PB1₄₁₃₋₄₂₁ peptide, M1₅₈₋₆₆ peptide, or no peptide. CD8⁺ T cells were plated at a 1:1 ratio with peptide pulsed T2 cells. FIG. 1B. Summary of IFNγ secreting CD8⁺ T cells determined using an ELISpot assay. The results are an average of triplicates with background subtracted. All data points are statistically significant (p<0.05), with a cutoff value of 5 SFC/100000 CD8⁺ T cells.

FIGS. 2A-B. aAPC induce antigen-specific immunodominant and subdominant CD8⁺ T cells. Legend: CD8⁺ T cells were stimulated weekly for 3 weeks with M1₅₈₋₆₆-aAPC (left panels). Cells were stimulated with pool 1 aAPC, which included NA₂₃₁₋₂₃₉-aAPC, PA₂₂₅₋₂₃₃-aAPC, and PB1₄₁₃₋₄₂₁-aAPC (middle panels). Cells were stimulated weekly with pool 2 aAPC, which included NA₇₅₋₈₄-aAPC, PA₄₆₋₅₄-aAPC, and NS1₁₂₃₋₁₃₂-aAPC (right panels). FIG. 2A. aAPC generated specific CD8⁺ T cells from adult donor C1 after 3 rounds of stimulation and analyzed by flow cytometry after staining with either HLA A2-Ig or A2-tetramer. Cells were stained with, I. noncognate, MART-1 loaded tetramer for background, II. cognate, M1₅₈₋₆₆ loaded tetramer, III and V. unloaded A2-Ig dimer, IV. cognate, PB1₄₁₃₋₄₂₁ loaded A2-Ig dimer, VI. cognate, PA₄₆₋₅₄ loaded A2-Ig dimer. FIG. 2B. IFNγ production by aAPC stimulated CD8⁺ T cells from donor C1 after 3 rounds of stimulation as determined by ICS. I, III, V. Cells were stimulated with unpulsed T2 cells at a 1:1 ratio. II CD8⁺ T cells were stimulated with M1₅₈₋₆₆ pulsed T2 cells. IV. CD8⁺ T cells were stimulated with PB1₄₁₃₋₄₂₁ pulsed T2 cells. VI. CD8⁺ T cells are stimulated with PA₄₆₋₅₄ pulsed T2 cells.

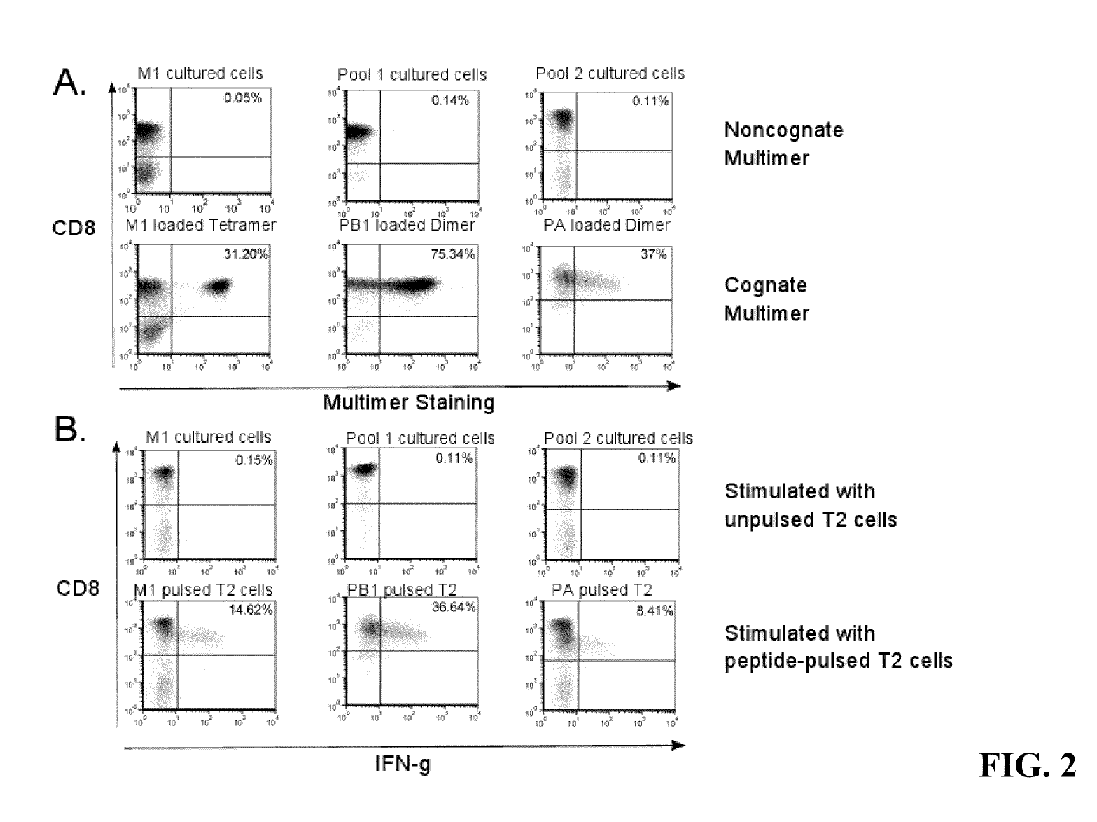

FIGS. 3A-C. Control donors' CD8⁺ T cell response to influenza A epitopes is broad and donor specific. FIG. 3A. IFNγ secretion from control donors after 3 weeks of aAPC stimulation as determined by ICS and analyzed by flow cytometry. M1 is the immunodominant epitope. A positive result is defined as 5 fold above background. Background levels were determined by stimulating CD8⁺ T cells with unpulsed T2 cells, as described in FIG. 2. FIG. 3B. Comparison of the immunodominant response in control donors. The percent of positive donors for IFN γ secretion by ICS after 3 weeks of aAPC stimulation is compared to the percent of positive donors for IFN γ secretion by ELISpot at wk 0 from control donors. FIG. 3C. Comparison of aAPC based stimulation to ELISpot assays for the immunodominant and subdominant influenza-specific responses. The number of positive responses to subdominant epitopes per control donor is graphed. Positive responses for aAPC expansion were determined by IFNγ secretion by ICS after three weeks of aAPC stimulation. The mean number of subdominant responses per control donor by aAPC stimulation was 3.3. The mean number of subdominant responses per donor by ELISpot was 1.3. This difference has a p=0.044. A donor is considered positive by ICS if the IFNγ secretion was 5 fold or greater over background. A donor is considered positive by ELISpot if the number of SFC is greater than 5, with a p<0.05.

FIG. 4. Geriatric donors' CD8⁺ T cell response to influenza A epitopes is limited. Legend: A) IFNγ secretion from geriatric donors after 3 weeks of aAPC stimulation as determined by ICS and analyzed by flow cytometry. M1 is the immunodominant epitope. Background levels were determined by stimulating CD8⁺ T cells with unpulsed T2 cells, as described in FIG. 2.

FIGS. 5A-B. Alterations in the CTL immune response to subdominant influenza epitopes in the population. FIG. 5A. Comparison of CTL response between control donors and geriatric donors based on IFNγ secretion by ICS after three weeks of aAPC stimulation. Filled bars are control donors, lined bars are geriatric donors. FIG. 5B. Comparison of the number of responses to subdominant epitopes per donor, comparing control to geriatric donors. Positive responses were determined by IFNγ secretion by ICS after three weeks of aAPC stimulation. The mean number of subdominant responses per control donor was 2.8. The mean number of subdominant responses per geriatric donor was 0.44. This difference has a p=0.0008.

FIGS. 6A-B. Schematic of aAPC production and aAPC based stimulation assay. FIG. 6A. A2-Ig based aAPC were prepared by attaching A2-Ig dimer and anti-CD28 antibody to epoxy beads at a 1:1 ratio. A2-Ig molecules were loaded with peptide. aAPC beads were pulsed with one peptide being loaded onto A2-Ig aAPC per vial. FIG. 6B. CD8⁺ T cells were cultured with peptide-pulsed aAPC. They were plated with a single peptide loaded aAPC or with a pool of 3 aAPC each individually loaded with respective peptide. The ratio of CD8⁺ T cells to aAPC remained constant at 1:1.

FIGS. 7A-B. aAPC induce influenza-specific subdominant cells from memory CD8⁺ T cells. Legend: Memory and naïve CD8⁺ T cells were separated and then cultured with the aAPC based stimulation assay. CD8⁺ T cells were stimulated weekly for 3 weeks with pool 1 aAPC, which included NA₂₃₁₋₂₃₉-aAPC, PA₂₂₅₋₂₃₃-aAPC, and PB1₄₁₃₋₄₂₁-aAPC (left panels). Cells were stimulated weekly with pool 2 aAPC, which included NA₇₅₋₈₄-aAPC, PA₄₆₋₅₄-aAPC, and NS1₁₂₃₋₁₃₂-aAPC, (right panels). FIG. 7A. IFNγ production by aAPC stimulated memory CD8⁺ T cells from donor C1 as determined by ICS. I, III. Cells were stimulated with unpulsed T2 cells at a 1:1 ratio. II. CD8⁺ T cells were stimulated with PB1₄₁₃₋₄₂₁ pulsed T2 cells. IV. CD8⁺ T cells are stimulated with PA₄₆₋₅₄ pulsed T2 cells. FIG. 7B. IFNγ production by aAPC stimulated naïve CD8⁺ T cells from donor C1 as determined by ICS. I, III. Cells were stimulated with unpulsed T2 cells at a 1:1 ratio. II, IV. CD8⁺ T cells were stimulated with PB1₄₁₃₋₄₂₁ pulsed T2 cells. IV. CD8⁺ T cells are stimulated with PA₄₆₋₅₄ pulsed T2 cells.

### DETAILED DESCRIPTION

To assess the breadth and depth of influenza-specific immune responses, we compared ELISpot analysis to artificial Antigen Presenting Cells (aAPC)-based stimulation. Methods of making aAPCs are described in US2004/0115216 and in the Examples, below. We found that the repetitive aAPC-based stimulation assay was a more sensitive method to detect the breadth of influenza-specific responses. Using the aAPC assay to stimulate influenza-specific CD8⁺ T cells ex vivo from healthy, control donors, aged 21-42, and geriatric donors, over the age of 65, we found that both control and geriatric donors had an immunodominant M1₅₈₋₆₆ CTL response, however, geriatric donors lacked a broad, multi-specific response to subdominant epitopes seen in the control donors. This was due to changes in memory CD8⁺ T cell responses; i.e., aging leads to a decrease in the subdominant influenza-specific CTL responses which may contribute to the increased morbidity and mortality in older individuals.

This disclosure therefore provides compositions that can be used to induce an immune response against an influenza virus, for example in elderly populations. These compositions and methods of administering them are described below.

In addition, the sensitive aAPC-based stimulation assay described in the Examples below can be used to identify populations of individuals who lack responses to epitopes of other infectious agents. Immunogenic compositions can then be tailored to induce broad, multi-specific immune responses to subdominant epitopes of other infectious agents, such as protozoa, bacteria, fungi (both unicellular and multicellular), viruses, prions, intracellular parasites, helminths, and other infectious agents that can induce an immune response.

Bacterial antigenic peptides include those of gram-positive cocci, gram positive bacilli, gram-negative bacteria, anaerobic bacteria, such as organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaceae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma.

Antigenic peptides of protozoan infectious agents include those of malarial plasmodia, Leishmania species, Trypanosoma species and Schistosoma species.

Fungal antigenic peptides include those of Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, and Malassezia.

Antigenic peptides of prions include those of the sialoglycoprotein PrP 27-30 of the prions that cause scrapie, bovine spongiform encephalopathies (BSE), feline spongiform encephalopathies, kuru, Creutzfeldt-Jakob Disease (CJD), Gerstmann-Strassler-Scheinker Disease (GSS), and fatal familial insomnia (FFI).

Intracellular parasites from which antigenic peptides can be obtained include, but are not limited to, Chlamydiaceae, Mycoplasmataceae, Acholeplasmataceae, Rickettsiae, and organisms of the genera Coxiella and Ehrlichia.

Antigenic peptides can be obtained from helminths, such as nematodes, trematodes, or cestodes.

Viral antigenic peptides include, but are not limited to, those of adenovirus, herpes simplex virus, papilloma virus, respiratory syncytial virus, poxviruses, HIV, influenza viruses, and CMV. Particularly useful viral peptide antigens include HIV proteins such as HIV gag proteins (including, but not limited to, membrane anchoring (MA) protein, core capsid (CA) protein and nucleocapsid (NC) protein), HIV polymerase, influenza virus matrix (M) protein and influenza virus nucleocapsid (NP) protein, hepatitis B surface antigen (HBsAg), hepatitis B core protein (HBcAg), hepatitis e protein (HBeAg), hepatitis B DNA polymerase, hepatitis C antigens, and the like.

### Compositions for Inducing Influenza-Specific Immune Responses

Immunogenic compositions typically comprise two or more purified peptides selected from the group consisting of NMLSTVLGV (SEQ ID NO:2), CVNGSCFTV (SEQ ID NO:3), SLENFRAYV (SEQ ID NO:4), IMDKNIILKA (SEQ ID NO:5), SLCPIRGWAI (SEQ ID NO:6), and FMYSDFHFI (SEQ ID NO:7). In some embodiments the composition comprises three, four, five, or six purified peptides. Optionally, other antigenic peptides can be included in the composition, such as GILGFVFTL (SEQ ID NO:1). Combinations of peptides that can be included in a composition include, but is not limited to, combinations of peptides listed in the following table.

SEQ ID NOS: 2, 3
SEQ ID NOS: 2, 4
SEQ ID NOS: 2, 5
SEQ ID NOS: 2, 6
SEQ ID NOS: 2, 7
SEQ ID NOS: 3, 4
SEQ ID NOS: 3, 5
SEQ ID NOS: 3, 6
SEQ ID NOS: 3, 7
SEQ ID NOS: 4, 5
SEQ ID NOS: 4, 6
SEQ ID NOS: 4, 7
SEQ ID NOS: 5, 6
SEQ ID NOS: 5, 7
SEQ ID NOS: 6, 7
SEQ ID NOS: 2, 3, 4
SEQ ID NOS: 2, 3, 5
SEQ ID NOS: 2, 3, 6
SEQ ID NOS: 2, 3, 7
SEQ ID NOS: 2, 4, 5
SEQ ID NOS: 2, 4, 6
SEQ ID NOS: 2, 4, 7
SEQ ID NOS: 2, 5, 6
SEQ ID NOS: 2, 5, 7
SEQ ID NOS: 2, 6, 7
SEQ ID NOS: 3, 4, 5
SEQ ID NOS: 3, 4, 6
SEQ ID NOS: 3, 4, 7
SEQ ID NOS: 3, 5, 6
SEQ ID NOS: 3, 5, 7
SEQ ID NOS: 3, 6, 7
SEQ ID NOS: 4, 5, 6
SEQ ID NOS: 4, 5, 7
SEQ ID NOS: 5, 6, 7
SEQ ID NOS: 2, 3, 4, 5
SEQ ID NOS: 2, 3, 5, 6
SEQ ID NOS: 2, 3, 6, 7
SEQ ID NOS: 2, 3, 4, 7
SEQ ID NOS: 2, 4, 5, 6
SEQ ID NOS: 2, 4, 6, 7
SEQ ID NOS: 3, 4, 5, 6
SEQ ID NOS: 4, 5, 6, 7
SEQ ID NOS: 2, 3, 4, 6
SEQ ID NOS: 2, 3, 5, 7
SEQ ID NOS: 2, 3, 4, 7
SEQ ID NOS: 2, 4, 5, 7
SEQ ID NOS: 3, 4, 5, 7
SEQ ID NOS: 3, 4, 5, 6, 7
SEQ ID NOS: 2, 4, 5, 6, 7
SEQ ID NOS: 2, 3, 5, 6, 7
SEQ ID NOS: 2, 3, 4, 6, 7
SEQ ID NOS: 2, 3, 4, 5, 7
SEQ ID NOS: 2, 3, 4, 5, 6

Peptides that are described herein may be synthesized using any known method in the art. The redundancy of the genetic code is well-known. Thus, any nucleic acid molecule (polynucleotide) which encodes one of the disclosed peptides can be used to produce that peptide recombinantly. Nucleic acid molecules can be synthesized using an amplification technique, such as the polymerase chain reaction (PCR), or by using an automatic synthesizer. See Caruthers et al., Nucl. Acids Res. Symp. Ser. 215, 223, 1980; Horn et al., Nucl. Acids Res. Symp. Ser. 225, 232, 1980; Hunkapiller et al., Nature 310, 105-11, 1984; Grantham et al., Nucleic Acids Res. 9, r43-r74, 1981.cDNA molecules can be made with standard molecular biology techniques, using mRNA as a template. cDNA molecules can thereafter be replicated using molecular biology techniques well known in the art. An amplification technique, such as PCR, can be used to obtain additional copies of polynucleotides of the invention, using either genomic DNA or cDNA as a template.

A nucleic acid molecule which encodes one or more of the disclosed peptides can be inserted into an expression vector which contains the necessary elements for the transcription and translation of the inserted coding sequence. Methods which are well known to those skilled in the art can be used to construct expression vectors containing coding sequences and appropriate transcriptional and translational control elements. These methods include in vitro recombinant DNA techniques, synthetic techniques, and in vivo genetic recombination. Host cells for producing the disclosed peptides can be prokaryotic or eukaryotic (e.g., E. coli; yeasts; baculovirus; mammalian cells). Expression constructs can be introduced into host cells using well-established techniques which include, but are not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun” methods, and DEAE- or calcium phosphate-mediated transfection.

In other embodiments, peptides are synthesized, for example, using solid phase techniques. See, e.g., Merrifield, J. Am. Chem. Soc. 85, 2149 54, 1963; Roberge et al., Science 269, 202 04, 1995. Protein synthesis can be performed using manual techniques or by automation. Automated synthesis can be achieved, for example, using Applied Biosystems 431A Peptide Synthesizer (Perkin Elmer).

In some embodiments peptides are present individually in a composition. In other embodiments, a composition may contain a fusion polypeptide containing one or more peptides. Methods of making such fusion polypeptides are well known in the art and are described, for example, in US2010/0068218.

In other embodiments the peptides are administered to the individual in a composition comprising one or more nucleic acid constructs that encode the peptides.

The disclosed compositions can be administered to any individual in whom it is desired to induce an immune response against an influenza virus, including pediatric individuals, children, young adults, adults, and the elderly. In certain embodiments the individual is at least 65 years old. In some embodiments the individual is over 65, 66, 67, 68, 69, 70, 71, 72, 73, 74, 75, 76, 77, 78, 79, or 80 years old. In other embodiments the individual is over 81, 82, 83, 84, 85, 86, 87, 88, 87, or 90 years old. In other embodiments the individual is over 91, 92, 93, 94, or 95 years old.

Typically, an effective amount of the composition is administered. An “effective amount” of an immunogenic composition is the amount that induces a detectable immune response in the individual. When the composition is administered as a vaccine composition, an effective amount is the amount sufficient to detectably reduce a symptom of influenza or to reduce the risk of acquiring an influenza infection.

In some embodiments the composition is administered by parenteral administration, e.g., by injection via the intradermal, intravenous, intramuscular, subcutaneous, or intraperitoneal routes). In other embodiments the composition is administered topically directly to the mucosa, for example by nasal drops or mist, inhalation, or by nebulizer. Nasal aerosols or mists are typical means of topical administration.

Some variation in dosage and regimen will necessarily occur depending on the age and medical condition of the subject being treated, as well as the route chosen. The person responsible for administration will, in any event, determine the appropriate dose for the individual subject. Doses can be, for example, between 10 μg/kg and 1 mg/kg (e.g. between 30 μg/kg and 500 μg/kg, between 50 μg/kg and 250 μg/kg, or between 100 μg and 200 μg).

In many instances, it will be desirable to have multiple administrations of the vaccine. Thus, one or more disclosed compositions may be administered 1, 2, 3, 4, 5, 6, 7, 8, 9, 10, or more times. The administrations will normally be at from one to twelve week intervals, more usually from one to six week intervals. Periodic re-administration will be desirable with recurrent exposure to the pathogen.

Compositions typically contain a pharmaceutically acceptable vehicle; i.e., a vehicle that does not produce an allergic or other adverse reaction in the individual to whom it is administered. Preparation of aqueous injectable compositions comprising peptides as the active ingredient are well understood in the art.

In some embodiments the composition further comprises an adjuvant, such as one or more of a mineral salt (e.g., aluminum hydroxide, aluminum aluminium phosphate, or calcium phosphate); oil emulsions (e.g., MF59); particulate adjuvants (e.g., virosomes, structured complex of saponins and lipids or ISCOMS); microbial derivatives (e.g., monophosphoryl lipid A); CpG motifs; modified toxins; plant derivatives (e.g., saponins); and endogenous immunostimulatory adjuvants (e.g., cytokines).

In certain embodiments, it may prove useful to use the disclosed immunogenic compositions in conjunction with an anti-viral therapy. The well known two classes of anti-virals are neuraminidase inhibitors and M2 inhibitors (adamantane derivatives). Anti-viral drugs such as oseltamivir (TAMIFLU®) and zanamivir (RELENZA®) are neuraminidase inhibitors that are often effective against both influenza A and B. The anti-viral drugs amantadine and rimantadine are designed to block a viral ion channel (M2 protein) and prevent the virus from infecting cells. These drugs are sometimes effective against influenza A if given early in the infection but are always ineffective against influenza B.

The above disclosure generally describes the present invention. A more complete understanding can be obtained by reference to the following specific examples, which are provided for purposes of illustration only and are not intended to limit the scope of the invention.

### Example 1

### Materials and Methods

Donors' Demographics

All donors were HLA-A2⁺ as typed by monoclonal antibody BB7.2. All donors in the control group were between the ages of 21-42 (Table 2). The group consisted of both males and females, many of whom had previously received an influenza vaccine. All geriatric donors were 67 and above (Table 3). They were also a mixed group of males and females and varied in the timing of their last influenza vaccine. All elderly donors were healthy. All donors gave informed consent before enrolling in the study. The Institutional Review Boards of Johns Hopkins Medical Institutions, Case Western Reserve University, and the Cleveland VA approved this investigation.

TABLE 2
Demographic characteristics of control donors.

| Donor | Age | Sex | Last year of Flu vaccine |
|---|---|---|---|
| C1 | 25 | M | Never |
| C2 | 27 | F | 2007 |
| C3 | 25 | M | Never |
| C4 | 42 | M | 2007 |
| C5 | 34 | F | 2008 |
| C6 | 31 | M | 2007 |
| C7 | 30 | M | 2007 |
| C8 | 30 | M | 2007 |
| C9 | 35 | M | 2008 |
| C10 | 32 | M | >5 years ago |
| C11 | 26 | M | 2009 |
| C12 | 32 | M | 2009 |

TABLE 3
Demographic characteristics of geriatric donors

| Donor | Age | Sex | Last year of Flu vaccine |
|---|---|---|---|
| E1 | 83 | M | 2007 |
| E2 | 83 | F | 2007 |
| E3 | 68 | M | 2008 |
| E4 | 67 | F | >5 years ago |
| E5 | 72 | M | 2008 |
| E6 | 70 | M | 2008 |
| E7 | 86 | M | >3 years ago |
| E8 | 86 | M | 2009 |
| E9 | 90 | | |

Peripheral Blood Mononuclear Cells (PBMC)

Blood was obtained from donors using VacutainerCPT cell preparation tubes or heparin green tops (Becton-Dickinson) PBMC were isolated by Ficoll-Hypaque (Amersham Pharmacia Biotek, Uppsala, Sweden) density gradient centrifugation. CD8⁺ primary human T cells were isolated from the PBMC using the untouched human CD8⁺ T cell isolation kit (Miltenyi). Naïve cells were further selected by secondary enrichment with naïve CD8⁺ T cell isolation kit (Miltenyi).

Cell Lines

TAP (transporter associated with antigen processing)-deficient 174CEM.T2 (T2) cells were maintained in M′ medium (RPMI 1640 medium (Gibco, Invitrogen Corporation), non-essential amino acids (Sigma-Aldrich), sodium pyruvate (Gibco, Invitrogen Corporation), vitamin solution (Gibco), 2-mercaptoethanol (Gibco), 10 μM ciprofloxacin (Serologicals Proteins Inc)) supplemented with 10% fetal calf serum (Atlanta Biologicals).

Peptides

All peptides M1₅₈₋₆₆: GILGFVFTL (SEQ ID NO:1), PB1₄₁₃₋₄₂₁: NMLSTVLGV (SEQ ID NO:2), PA₂₂₅₋₂₃₃: SLENFRAYV (SEQ ID NO:3), NA₂₃₁₋₂₃₉: CVNGSCFTV (SEQ ID NO:4), PA₄₆₋₅₂: FMYSDFHFI (SEQ ID NO:5), NA₇₅₋₈₄: SLCPIRGWAI (SEQ ID NO:6), and NS1₁₂₅₋₁₃₂: IMDKNIILKA (SEQ ID NO:7) were synthesized by GenScript (Table 1). Purity of all peptides (>95%) was confirmed by mass-spectral analysis and high-pressure liquid chromatography. Peptides were dissolved in dimethylsulfoxide (DMSO) and PBS for a final concentration of 1 mg/mL and sterile-filtered through a 0.22-μm SpinX (Corning).

TABLE 1
Influenza peptides separated into pools.

| Peptide name | Peptide Sequence | |
|---|---|---|
| Immunodominant | | |
| M1₅₈₋₆₆ | GILGFVFTL | (SEQ ID NO: 1) |
| Subdominant Peptides | | |
| Pool 1 | | |
| PB1₄₁₃₋₄₂₁^{a, b} | NMLSTVLGV | (SEQ ID NO: 2) |
| NA₂₃₁₋₂₃₉^{c} | CVNGSCFTV | (SEQ ID NO: 3) |
| PA₂₂₅₋₂₃₃^{a, b} | SLENFRAYV | (SEQ ID NO: 4) |
| Subdominant Peptides | | |
| Pool 2 | | |
| NS1₁₂₃₋₁₃₂^{d} | IMDKNIILKA | (SEQ ID NO: 5) |
| NA₇₅₋₈₄^{a, b} | SLCPIRGWAI | (SEQ ID NO: 6) |
| PA₄₆₋₅₄^{a, b} | FMYSDFHFI | (SEQ ID NO: 7) |

^{a}Peptides were selected from (22)
^{b}Indicates the peptide's binding affinity to HLA-A2 has been published by Gianfrani et al. (22)
^{c}Peptides were selected from (39)
^{d}Peptides were selected from (40)

ELISpot Assay

Polyvinylidene fluoride (PVDF) membrane-bottomed 96 well plates (Millipore) were coated with anti-IFNγ monoclonal antibody (EBiosciences) overnight at 4° C. T2 cells were pulsed with 10 μg/mL peptide in serum-free M′ media overnight at 37° C. Plates were washed with ELISpot coating buffer (EBiosciences) and blocked with RPMI-1640 supplemented with 10% FBS (Gibco) for one hour at room temperature. T2 cells were harvested and washed two times with M′ media. CD8⁺ T cells were isolated from PBMCs as described above. Effector and target cells were plated at a 1:1 ratio in the IFNγ coated PVDF 96 well plate. Negative control wells contained unpulsed T2 cells with effector cells. The plates were incubated at 37° C. for 16-20 hrs. The plates were washed two times with ELISpot wash buffer (PBS, 0.1% TWEEN® 20) (Gibco, Sigma-Aldrich) and incubated with secondary anti-IFNγ mAb (EBiosciences) for 2 hours at room temperature or overnight at 4° C. Plates were washed with ELISpot wash buffer and incubated with avidin horseradish peroxidase enzyme complex (EBiosciences) for 45 minutes at 4° C. Plates were developed using AEC peroxidase substrate (Sigma-Aldrich). Colored spot-forming cells (SFC) were counted using an automated ELISpot reader (Immunospot, CellularTechnology). A two-tailed T test was used to determine statistical significance.

Generation of Artificial Antigen Presenting Cells

A2-Ig based aAPC was prepared according to the previously described method (24). A 1:1 mixture of A2-Ig and anti-CD28 monoclonal antibody 9.3 (courtesy of Carl June, University of Pennsylvania) was added to 0.5 ml of washed epoxy beads (DYNABEADS®, M-450, Epoxy, 4×10⁸ beads/ml) (Dynal) in sterile 0.1 M Borate buffer, pH 7.0-7.4. The beads were rotated for 24 h at 4° C. Then the beads were washed twice with bead wash buffer and the protein expression on the bead was checked by flow cytometry. Next, A2-Ig molecules were loaded with 30 μg/ml of a single peptide (GenScript) in 1 ml PBS containing 5×10⁷ beads and rotated overnight at 4° C. aAPC beads were stored in peptide solution at 4° C., with only a single peptide being loaded onto individual A2-Ig aAPC per vial. aAPCs were pulsed with M1₅₈₋₆₈ peptide (M1₅₈₋₆₈-aAPC), NA₂₃₁₋₂₃₉ peptide (aAPCsNA₂₃₁₋₂₃₉-aAPC), PA₂₂₅₋₂₃₃ peptide (PA₂₂₅₋₂₃₃-aAPC), PB1₄₁₃₋₄₂₁ peptide (PB1₄₁₃₋₄₂₁-aAPC), NA₇₅₋₈₄ peptide (NA₇₅₋₈₄-aAPC), PA₄₆₋₅₄ peptide (PA₄₆₋₅₄-aAPC), and NS1₁₂₃₋₁₃₂ peptide (NS1₁₂₃₋₁₃₂-aAPC).

Expansion of Primary Human CD8⁺ T Cells

CD8⁺ T cells (10⁶/plate) were co-cultured at a 1:1 ratio with peptide-loaded aAPC in a 96-well round-bottom plate (BD Falcon) with 165 μl/well M′ medium, supplemented with 5% autologous plasma or 5% Human AB serum (HyClone) and 6% T-cell growth factor (TCGF) at 37° C. in a 5% CO₂ incubator. TCGF was prepared as previously described (37). The culture media was replenished once a week on day 4. On day 7 CD8⁺ T cells were harvested, counted and re-plated at 10⁶ CD8⁺ T cells per 96 well plate with 10⁶ fresh peptide-loaded aAPC. This was repeated weekly for up to 5 weeks. For the immunodominant M1₅₈₋₆₆ generated CTL, cells were plated at a 1:1 ratio with only M1₅₈₋₆₈-aAPC. For the subdominant epitopes, T cells were cultured at a 1:1 ratio of T cells to aAPC where the aAPC consisted of a pool of aAPC. Pool 1 consisted of NA₂₃₁₋₂₃₉-aAPC, PA₂₂₅₋₂₃₃-aAPC, and PB1₄₁₃₋₄₂₁-aAPC. Pool 2 consisted of NA₇₅₋₈₄-aAPC, PA₄₆₋₅₄-aAPC, and NS1₁₂₃₋₁₃₂-aAPC (Table 1). All aAPC were peptide loaded individually and then pooled when added to the plates with the T cells.

Multimer Staining and Flow Cytometric Analysis

The antigen specificity of the CTL was tested by staining with anti-human CD8 FITC monoclonal antibody (clone UCHT-4, Sigma-Aldrich) and HLA-A2 tetramer PE loaded with either Mart-1 peptide (Mart-1 tetramer) for noncognate control, M1₅₈₋₆₆ peptide (M1₅₈₋₆₆ tetramer) (Beckman Coulter Inc., San Diego, Calif.), or A2-Ig dimer loaded with PB1₄₁₃₋₄₂₁ peptide (PB1₄₁₃₋₄₂₁ Dimer), PA₂₂₅₋₂₃₃ peptide (PA₄₂₂₅₋₂₃₃ Dimer), NA₂₃₁₋₂₃₉ (NA₂₃₁₋₂₃₉ Dimer), PA₄₆₋₅₂ (PA₄₆₋₅₂ Dimer), NA₇₅₋₈₄ (NA₇₅₋₈₄ Dimer), or NS1₁₂₅₋₁₃₂ (NS1₁₂₅₋₁₃₂ Dimer). The noncognate dimer control was unloaded A2-Ig Dimer. All A2-Ig dimer was prepared in our laboratory (38). Cells were incubated with dimer for 45 min at 4° C. Cells were washed and incubated for 10 minutes with secondary antibody, anti-IgG1 PE antibody (Caltag). Cells were washed and incubated for 10 minutes with anti-CD8 FITC antibody (clone UCHT-4, Sigma-Aldrich). Cells were stained with peptide loaded-tetramer or dimers for 30 minutes at 4° C. Cells were washed and incubated for 10 minutes with anti-CD8 FITC antibody (clone UCHT-4, Sigma-Aldrich). Samples were collected using a FACS Calibur flow cytometer with CELLquest software and were analyzed using FCS Express software.

Intracellular Cytokine Staining and CD107a Assay

aAPC (10⁵/well) generated CTLs were placed in a single well of a 96-well flat-bottom plates (BD Falcon) at a 1:1 ratio with peptide pulsed or unpulsed T2 cells. Prior to stimulation, 10 μL α-human CD107a PE-Cy5 (BD Pharmingen, San Diego, Calif.) were added to each well. After 1 hour of incubation GolgiStop (BD Pharmingen) was added to each well. Cells were left to incubate for up to 10 hours, then samples were harvested, stained for with CD8 APC (BD Pharmingen), fixed and permeabilized with CytoPerm/CytoFix (BD Pharmingen), and stained for cytokines IFNγ FITC and IL-4 PE (BD Pharmingen) according to the manufacturer's protocol.

Statistical Analysis

Pairwise analysis was done between groups using the Mann-Whitney test. Statistical analysis was performed using a commercially available graphing and data analysis program (GraphPad Prism 5.01 for Windows, GraphPad Software, San Diego Calif. USA, www.graphpad.com). Significance was defined as p<0.05.

### Example 2

### Precursor Frequency of Influenza-Specific Cells

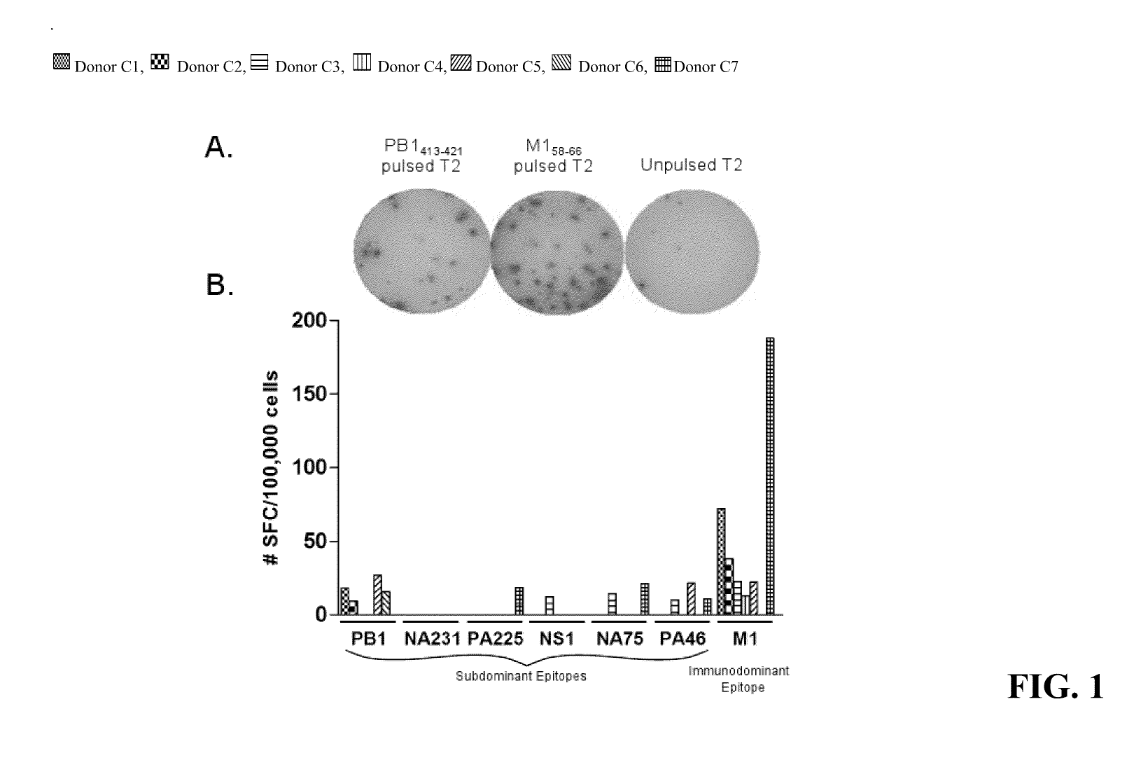

The precursor frequency of influenza-specific, immunodominant and subdominant, T cells was determined by an IFNγ ELISpot assay from seven control donors. Control donors were healthy and between the ages of 21-42. Similar to previously published data, we used the IFNγ ELISpot to determine the precursor frequency of immunodominant M1₅₈₋₆₆ specific CD8⁺ T cells directly ex vivo. We also analyzed the response to six subdominant influenza-specific peptides (Table 1). However, few donors had detectable CD8⁺ T cell precursor levels to multiple other influenza-specific subdominant epitopes (FIG. 1). Four out of the six donors showed a response to PB1₄₁₃₋₄₂₁, while only 2 donors responded to PA₄₆₋₅₄ and NA₇₅₋₈₄. One donor responded to NS1₁₂₃₋₁₃₂ or PA₂₂₅₋₂₃₃, and no donor responded to NA₂₃₁₋₂₃₉ (FIG. 1B). Based on this and other studies we estimated a limited subdominant repertoire in normal control donors (31).

### Example 2

### Stimulation of Subdominant Influenza-Specific CD8⁺ T Cells Using aAPC

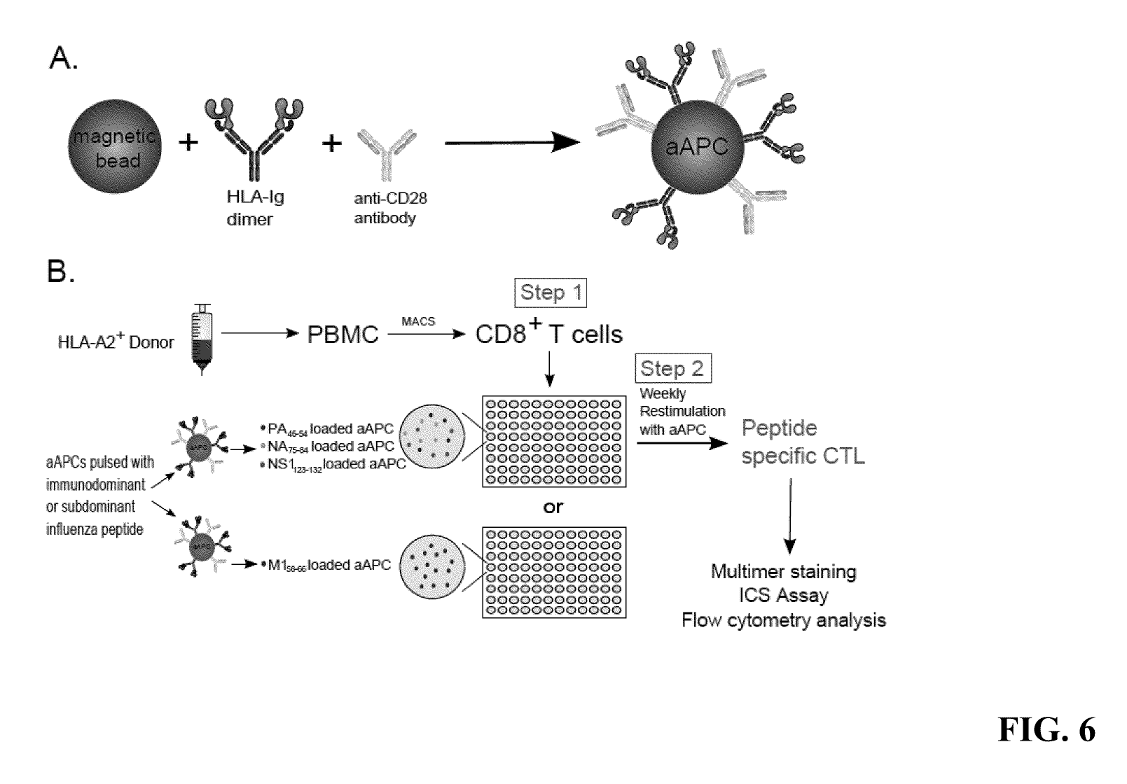

Since the precursor frequencies for the subdominant CTL specific response may be below the level of detection by ELISpot, we compared the ELISpot assay to an aAPC based stimulation assay initially designed for stimulation of viral CMV-immunodominant antigen-specific cells (33). Here, we tested if this approach would be useful in stimulating influenza subdominant-specific CD8⁺ T cells (FIG. 6). For these studies, we modified the original protocol by combining individually peptide-pulsed aAPC into pools of preloaded peptide-pulsed aAPC and stimulated purified CD8⁺ T cells with the pools of aAPC (Table 1). The pool of aAPC were plated at a 1:1 ratio to CD8⁺ T cells (FIG. 6). By using the pools of aAPC, as opposed to individually peptide-pulsed aAPC per stimulation, these experiments were logistically feasible with a modest, 40 cc, blood donation. After 3 rounds of weekly stimulation we analyzed our cultures by HLA-multimer staining and intracellular cytokine staining (ICS).

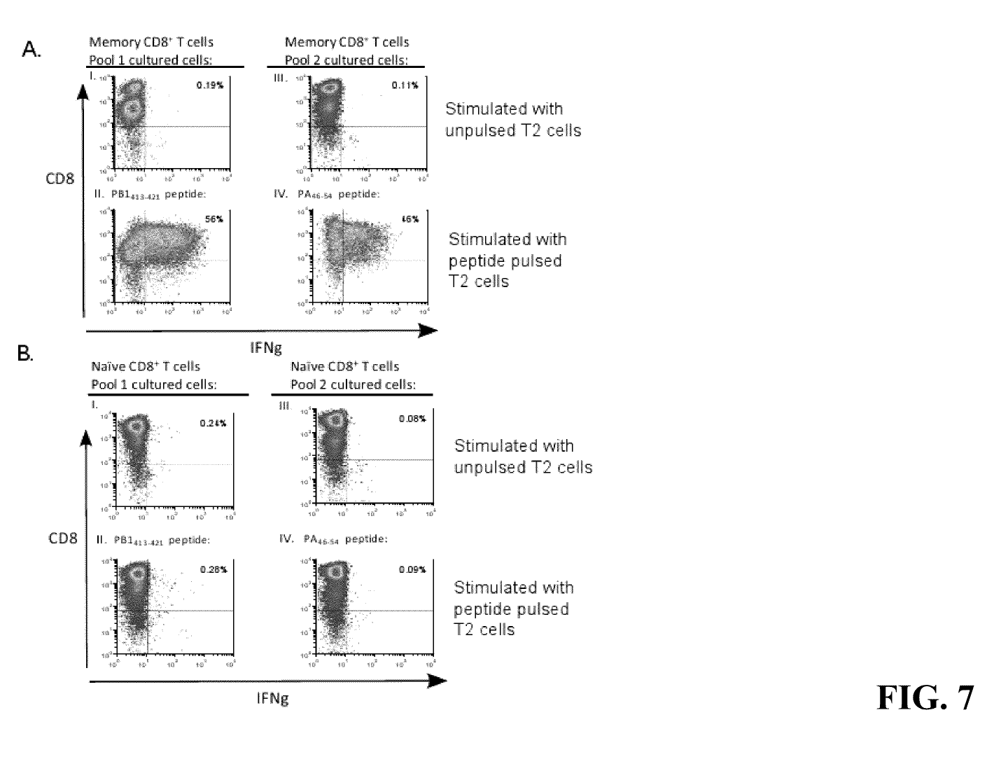

Using aAPC based stimulation, we were able to generate peptide specific CTLs against the immunodominant, M1₅₈₋₆₆ epitope, as well as the subdominant influenza-specific epitopes. Donor C1, a representative example, had approximately 31% M1-positive CD8⁺ T cells, based on M1₅₈₋₆₆ tetramer staining, and 15% M1₅₈₋₆₆-positive CD8⁺ T cells, based on IFNγ ICS after 3 weeks of culturing with M1₅₈₋₆₆ loaded aAPC (M1₅₈₋₆₈-aAPC) (FIGS. 7A, 7B, left panels). A fraction of the IFNγ⁺ population also coexpressed degranulation marker, CD107a.

aAPC also stimulated expansion of the subdominant epitope-specific CTLs. From the pool 1 aAPC cultures restimulated for three weeks, Donor C1's CD8⁺ T cells were 75% specific for the subdominant epitope PB1₄₁₃₋₄₂₁ by A2-Ig dimer staining and 37% specific by IFNγ expression (FIGS. 7A, 7B, middle panels). Similarly, antigen-specific CTL were obtained using aAPC loaded with pool 2 peptides. Donor C1 was 65% specific for PA₄₆₋₅₄ by A2-Ig dimer staining and 8% of the CD8⁺ T cells expressed IFNγ by ICS after three weekly stimulations (FIGS. 7A, 7B, right panel).

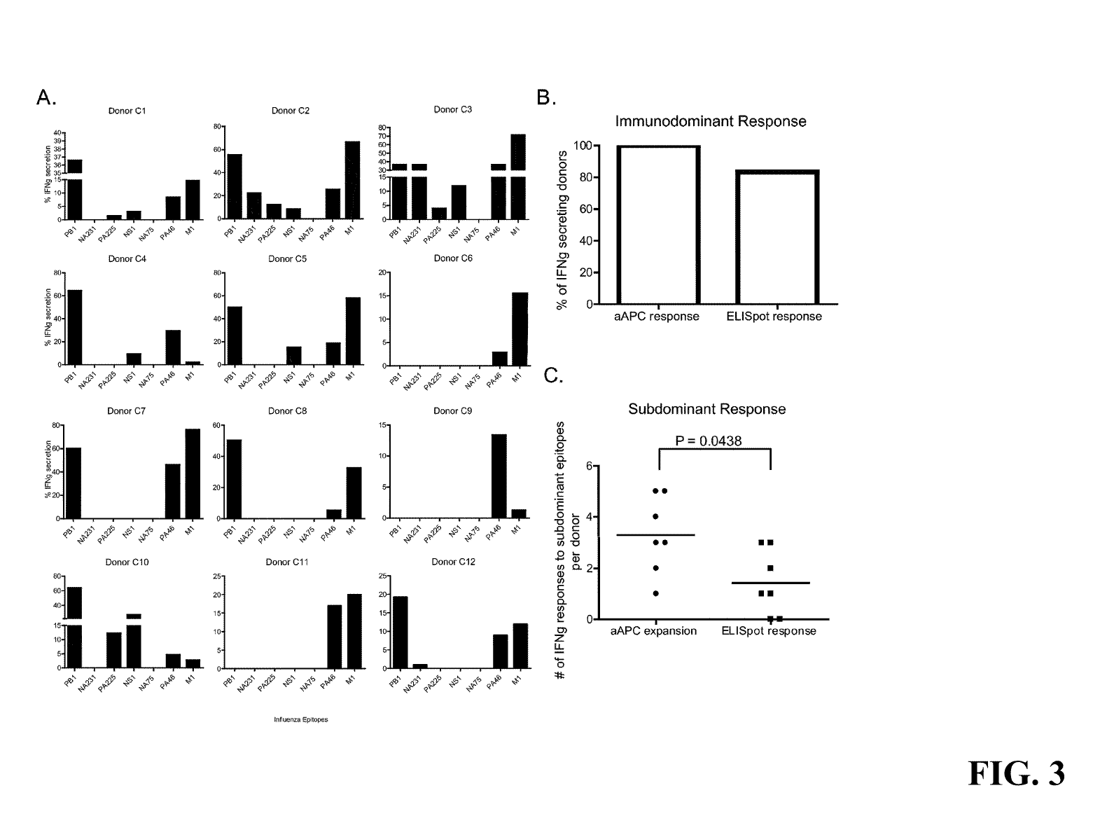

Pools of peptide-loaded aAPC were able to stimulate multiple antigen-specific T cell populations simultaneously. Depending on the donor, 2 or 3 different subdominant CTL responses could be seen within each pool. For example, pool 1 stimulated three different antigen-specific CTL from Donors C2 and C3, and pool 2 stimulated two antigen-specific CTL from the same donors (FIG. 3A).

### Example 3

### Comparison of ELISpot Versus aAPC Stimulation in Detecting Subdominant Influenza-Specific T Cells

aAPC based stimulation uniquely revealed responses not detected by ELISpot. Using single blood donations from seven donors, we compared the sensitivity of each assay. Both methods were comparable in detecting the immunodominant M1 specific responses; 100% of donors responded by aAPC stimulation and 6 out of 7, 83%, by ELISpot (FIG. 3B). However, the detection of subdominant specific T cells by aAPC expansion was significantly greater than their detection by ELISpot. The mean number of subdominant epitopes per donor detected by ELISpot was only 1.3 while the mean detected by aAPC assay was statistically higher at 3.3 (FIG. 3C).

### Example 4

### Influenza-Specific CD8⁺ T Cell Responses in Control Donors

Using the aAPC assay, we analyzed 12 control donors. aAPC based stimulation revealed broad CD8⁺ T cell responses to subdominant influenza-specific epitopes (FIG. 3A). Every donor had unique CD8⁺ T cell responses to at least one if not multiple subdominant epitopes. Several subdominant epitopes, PB1₄₁₃₋₄₂₁₅NS1₁₂₃₋₁₃₂, and PA₄₆₋₅₄, elicited a response from a majority of the donors. Furthermore, the breadth of the response to the subdominant and immunodominant epitopes varied between each donor (FIG. 3A). For standardization purposes, we analyzed all donors by ICS, however peptide-loaded HLA A2-Ig dimer staining revealed similar findings.

To determine if the aAPC based stimulation was expanding the memory CD8⁺ T cell population or the naïve population, we isolated naïve CD8⁺ T cells and memory CD8⁺ T cells from control donors. After three weeks of stimulation we found that the aAPC did not expand any subdominant specific cells from the naive CD8⁺ T cells. However the aAPC did expand subdominant specific cells from the memory CD8⁺ T cells (FIG. 7). This was independent of whether or not the donor showed a positive subdominant response by ELISpot.

### Example 5

### Influenza-Specific CD8⁺ T Cell Responses in Geriatric Donors

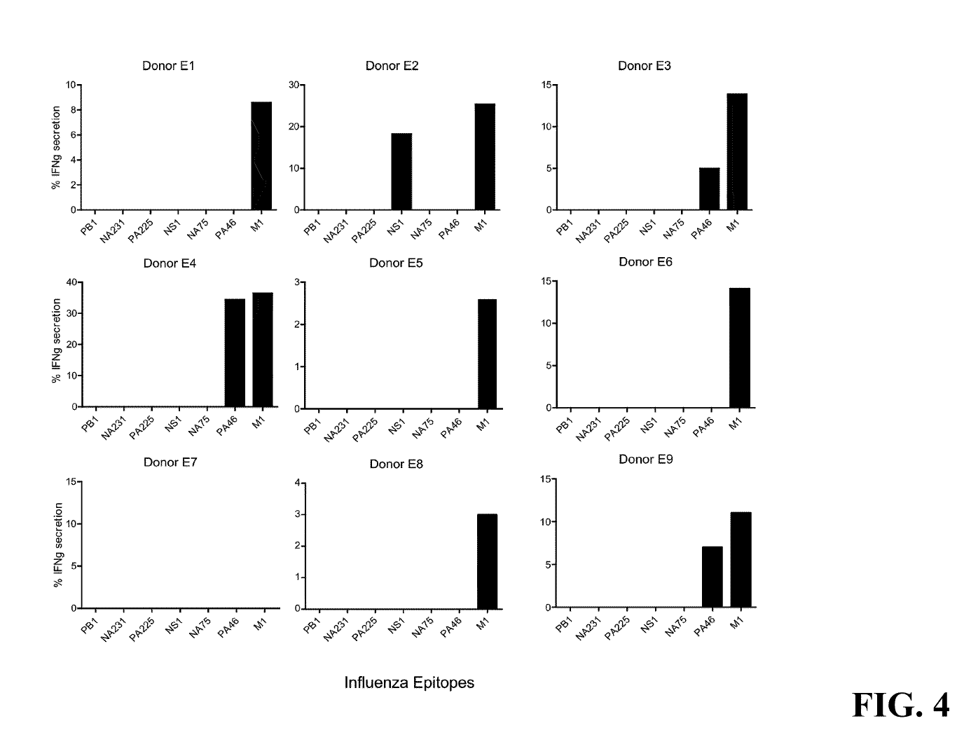

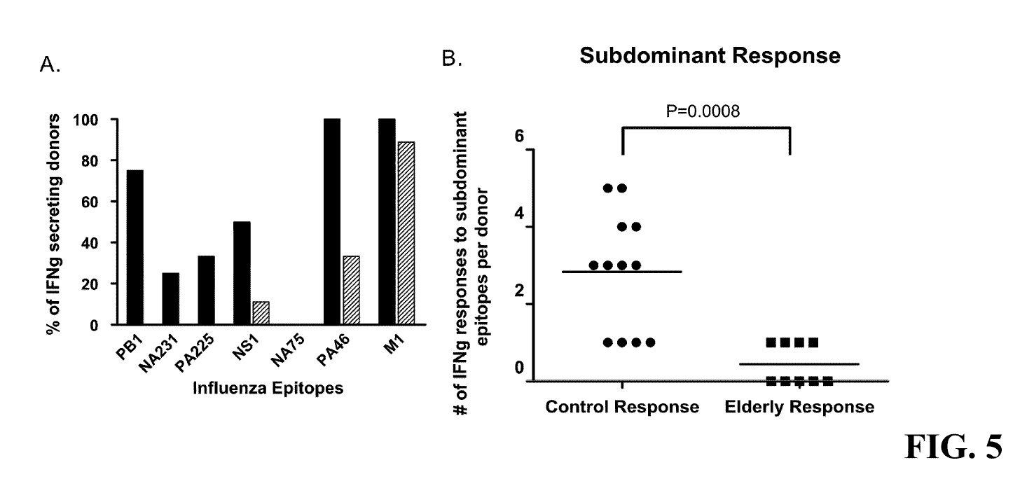

We sought to determine the breadth of influenza-specific CD8⁺ T cells in older adults aged 65 and above (Table 3). Eight of the nine donors in the elderly group had robust CD8⁺ T cells specific for the immunodominant M1₅₈₋₆₆ epitope (FIG. 4). However, geriatric donors lacked responses to most of the subdominant influenza peptides seen in the younger, control group (FIG. 4, FIG. 5). Of the donors who did elicit a subdominant response, the breadth of their response was limited compared to the control group (FIG. 4, FIG. 5). As noted earlier, in the control group, donors responded to as many as five of the six subdominant epitopes (FIG. 3A, FIG. 5). The mean number of subdominant epitopes per donor detected in the geriatric group was only 0.4, which was statistically different than the mean detected for control donors, 2.8 (p<0.0008). When a geriatric donor responded to a subdominant epitope, it was to a single subdominant epitope. None of the geriatric donors had multi-specific responses. Of note, Donors E2, E3, E4, and E8 responded to the subdominant epitopes NS1 or PA46, epitopes that were highly prevalent in the control group but none responded to another prevalent subdominant CTL epitope, PB1₄₁₃₋₄₂₁. (FIG. 3A, FIG. 4, FIG. 5A) Thus, in contrast to the broad response seen in control donors, there was a much more restricted response in geriatric donors.

Discussion

We have shown that pools of aAPC loaded with individual peptides can be used for expansion of multiple antigen specific T cell populations simultaneously. Furthermore, aAPC based stimulation was able to detect influenza-specific T cells that were below the limits of detection using a standard ELISpot assay. Using this novel technique, we were able to significantly enhance our ability to probe the breadth and depth of the human CD8⁺ T cell repertoire against influenza. In control donors, we found that all donors had T cells specific for the immunodominant peptide, and that many of the donors had T cells that were specific for multiple subdominant epitopes as well. Furthermore, we found that geriatric donors lacked the breadth of subdominant influenza-specific T cells, but maintained CD8⁺ T cells specific for the immunodominant, M1₅₈₋₆₆ peptide.

Our aAPC based stimulation assay overcomes some limitations associated with other assays. Precursor frequency analysis of peptide specific cells can be performed by a variety of sensitive assays including both ELISpot and multimer staining As with all assays they are limited by the precursor frequency of the events being analyzed and the background activity associated with each assay. Therefore, if the precursor frequency of peptide-specific T cells is below the level of background activity on the ELISpot assay, one would not be able to detect its presence. Multimer staining is often less sensitive than ELISpot. In the aAPC assay, CD8⁺ T cells are repetitively stimulated to expand antigen specific T cells to a level that is detectable by multiple methods, such as multimer staining, ICS or ELISpot. In the process of the repetitive stimulation we lose information on the exact precursor frequency, but the aAPC stimulation allowed detection of a wider range of antigen specific T cells that were previously undetectable.

Interestingly, the aAPC stimulation assay revealed that three of the six subdominant influenza epitopes, PB1₄₁₃₋₄₂₁, NS1₁₂₃₋₁₃₂, and PA₄₆₋₅₄, analyzed induce a response in the majority of the control donors. Since there are specific T cells against these subdominant epitopes in the majority of the donors, they do not meet the standard definition for subdominant responses (31) but are very low frequency events which were only effectively seen in the aAPC assay. It would be interesting to investigate why these epitopes are present at such low precursor frequencies, as their prevalence may implicate an important role in the CD8⁺ T cell immune response to influenza.

In contrast to the broad response seen in younger donors, there was a much more restricted response in geriatric donors. Our work is the first such broad-based analysis of changes in subdominant influenza-specific responses that occur with ageing. Our findings are consistent with published work that geriatric individuals maintain M1₅₈₋₆₆ specific CD8⁺ T cells (36-38), but extend that work significantly as we report that geriatric donors lack subdominant influenza-specific T cells even to the highly prevalent subdominant epitopes like PB1₄₁₃₋₄₂₁. These findings are also similar to recent animal studies that examine the impact of heterologous immunity on influenza CTL responses in ageing mice (39). Our studies both show a narrowing of the CD8⁺ T cell repertoire associated with aging.

The diminished immune response in geriatric individuals is often attributed to thymic involution, which leads to a reduction in the thymic output of naïve T cells (40). We wanted to determine if the lack of subdominant specific CD8⁺ T cells in the geriatric donors was due to a reduction in the naïve population or absence of the memory T cell population. Using control donors, we separated naïve CD8⁺ T cells from memory CD8⁺ T cells and were unable to expand any subdominant influenza-specific cells from the naïve CD8⁺ T cells using our aAPC (FIG. 7). Therefore, the lack of subdominant specific T cells in the geriatric group is likely due to alterations in the memory CD8⁺ T cell population and not a result of naïve CTL loss. Our data show the aAPC stimulation assay is expanding memory CD8⁺ T cells from donors, and the lack of subdominant specific T cells in the geriatric group is likely due to alterations in the memory CD8⁺ CTL population and not a result of naïve CTL loss. It has been suggested that adults maintain a naïve T cell repertoire through naïve CD8⁺ T cell homeostastis, as opposed to naïve T cells derived from the thymus (51). Considering this data, it is reasonable to assume, that like the geriatric donors, the control donors have limited production of naïve CD8⁺ T cells from the thymus, further supporting evidence that we are not priming naïve T cells with the aAPC assay.

Age-associated T cell repertoire changes have been previously reported. Changes in the functional T cell repertoire are known to occur in cytomegalovirus (CMV)-specific T cell responses in the geriatric population. Over time individuals lose their CD8⁺ T cell diversity and their immune response to CMV is narrowed (41-43). In contrast to influenza, CMV is a virus that persists latently in the body and the narrowing of the CD8⁺ T cell response is believed to be due to the persistence of CMV antigen throughout life. While influenza is not a latent virus, individuals may be frequently vaccinated and/or infected with influenza multiple times throughout life. It is possible that the multiple exposures lead to a shift in the T cell repertoire and a narrowing of the immune response that we observed in the geriatric population. Alternatively, the loss of the influenza-specific subdominant T cells in the geriatric donors might be a result of original antigenic sin. Original antigenic sin occurs when there are multiple infections with a similar, but not identical virus. The immune system is tricked into believing that the memory CD8⁺ T cells produced in the initial infection are sufficient to ward off the infection with a similar virus and this leads to a narrowing in the immune response (44, 45). Lastly, narrowing of the CTL response may be attributed to heterologous immunity. Heterologous immunity occurs when memory CD8⁺ T cells are activated during a secondary infection in response to a different virus than the memory cells were first directed against (46, 47). It is possible to imagine that other non-influenza viruses may induce responses where the immunodominant M1₅₈₋₆₆ specific CD8⁺ T cells are reactivated. These influenza-specific T cells are cross-reactive with the new, unrelated infection which leads to greater percentage of M1₅₈₋₆₆ specific CD8⁺ T cells, and a narrowing in the CTL immune response. Assuming that the control donors are representative of the geriatric donors at an earlier stage of their life, we see there is a shift in the memory CD8⁺ T cell population. Perhaps the subdominant specific CD8⁺ T cells are phenotypically and/or genotypically different from the immunodominant specific CD8⁺ T cells, and this may account for their loss while M1₅₈₋₆₆ specific T cells are maintained.

To summarize, we developed an aAPC stimulation assay that can be used for expansion and analysis of multiple antigen specific T cell populations simultaneously. Using this technique, we were able to significantly enhance our ability to probe the breadth and depth of the human CD8⁺ T cell repertoire against influenza. We found that all control donors had T cells specific for the immunodominant peptide, and that most had T cells that were specific for multiple subdominant epitopes as well. We found a significantly larger subdominant repertoire than expected in control donors and some of the subdominant responses were present in nearly all control donors. Compared to the broad responses seen in the younger control donors, there was near total absence of that response in the geriatric donors with maintenance of only the classical immunodominant M1-specific response. These results have potential implication for vaccine design targeted at boosting influenza-specific CD8⁺ T cells responses as it has been suggested that vaccine protection in geriatric donors correlates better with T cell responses than antibody responses (48). Therefore, understanding the mechanism that leads to the loss of subdominant influenza-specific CTLs may be crucial in designing a more effective vaccine for influenza and, more generally, for vaccines targeting the geriatric population.

### REFERENCE LIST

1. WorldHealthOrganization. EXPERTS TO DISCUSS BETTER PROTECTION AGAINST PANDEMIC INFLUENZA. Vol. 2009. Copenhagen; 2005.
2. Thompson W W, Shay D K, Weintraub E, et al. Mortality associated with influenza and respiratory syncytial virus in the United States. JAMA 2003; 289:179-186.
3. Thompson W W, Weintraub E, Dhankhar P, et al. Estimates of US influenza-associated deaths made using four different methods. Influenza Other Respi Viruses. 2009; 3:37-49.
4. Lamb R, Krug, R M. Fields Virology. Vol. 1 (ed 4th). Philadelphia: Lippincott Williams & Wilkins; 2001.
5. Bridges C B, Thompson W W, Meltzer M I, et al. Effectiveness and cost-benefit of influenza vaccination of healthy working adults: A randomized controlled trial. JAMA 2000; 284:1655-1663.
6. Potter J, Stott D J, Roberts M A, et al. Influenza vaccination of health care workers in long-term-care hospitals reduces the mortality of elderly patients. J Infect Dis. 1997; 175:1-6.
7. Daly K, Nguyen P, Woodland D L, Blackman M A. Immunodominance of major histocompatibility complex class I-restricted influenza virus epitopes can be influenced by the T-cell receptor repertoire. J. Virol. 1995; 69:7416-7422.
8. Gavazzi G, Krause K H. Ageing and infection. Lancet Infect Dis. 2002; 2:659-666.
9. Grubeck-Loebenstein B, Berger P, Saurwein-Teissl M, Zisterer K, Wick G. No immunity for the elderly. Nat. Med. 1998; 4:870.
10. Webster R G. Immunity to influenza in the elderly. Vaccine. 2000; 18:1686-1689.
11. Linton P J, Dorshkind K. Age-related changes in lymphocyte development and function. Nat. Immunol. 2004; 5:133-139.
12. Grubeck-Loebenstein B, Wick G. The aging of the immune system. Adv Immunol. 2002; 80:243-284.
13. Miller R A. Aging and immune function. Int Rev Cytol. 1991; 124:187-215.
14. Miller R A. The aging immune system: primer and prospectus. Science. 1996; 273:70-74.
15. Waldman R H, Mann J J, Small P A, Jr. Immunization against influenza. Prevention of illness in man by aerosolized inactivated vaccine. JAMA 1969; 207:520-524.
16. Doherty P C, Topham D J, Tripp R A, Cardin R D, Brooks J W, Stevenson P G. Effector CD4+ and CD8+ T-cell mechanisms in the control of respiratory virus infections. Immunol Rev. 1997; 159:105-117.
17. Lin Y L, Askonas B A. Biological properties of an influenza A virus-specific killer T cell clone. Inhibition of virus replication in vivo and induction of delayed-type hypersensitivity reactions. J Exp Med. 1981; 154:225-234.
18. Lukacher A E, Braciale V L, Braciale T J. In vivo effector function of influenza virus-specific cytotoxic T lymphocyte clones is highly specific. J Exp Med. 1984; 160:814-826.
19. Mackenzie C D, Taylor P M, Askonas B A. Rapid recovery of lung histology correlates with clearance of influenza virus by specific CD8+ cytotoxic T cells. Immunology. 1989; 67:375-381.
20. Kuwano K, Scott M, Young J F, Ennis F A. HA2 subunit of influenza A H1 and H2 subtype viruses induces a protective cross-reactive cytotoxic T lymphocyte response. J. Immunol. 1988; 140:1264-1268.
21. Kuwano K, Braciale T J, Ennis F A. Cytotoxic T lymphocytes recognize a cross-reactive epitope on the transmembrane region of influenza H1 and H2 hemagglutinins. Viral Immunol. 1989; 2:163-173.
22. Gotch F, McMichael A, Smith G, Moss B. Identification of viral molecules recognized by influenza-specific human cytotoxic T lymphocytes. J Exp Med. 1987; 165:408-416.
23. Bednarek M A, Sauma S Y, Gammon M C, et al. The minimum peptide epitope from the influenza virus matrix protein. Extra and intracellular loading of HLA-A2. J. Immunol. 1991; 147:4047-4053.
24. Morrison J, Elvin J, Latron F, et al. Identification of the nonamer peptide from influenza A matrix protein and the role of pockets of HLA-A2 in its recognition by cytotoxic T lymphocytes. Eur J. Immunol. 1992; 22:903-907.
25. Chisari F V, Ferrari C. Hepatitis B virus immunopathogenesis. Annu Rev Immunol. 1995; 13:29-60.
26. McMichael A J, Phillips R E. Escape of human immunodeficiency virus from immune control. Annu Rev Immunol. 1997; 15:271-296.
27. Rehermann B, Fowler P, Sidney J, et al. The cytotoxic T lymphocyte response to multiple hepatitis B virus polymerase epitopes during and after acute viral hepatitis. J Exp Med. 1995; 181:1047-1058.
28. Jameson H L, Arnott C H, Lillycrop K A. Regulation of herpes simplex virus immediate early gene expression. Biochem Soc Trans. 1998; 26:S258.
29. Assarsson E, Bui H H, Sidney J, et al. Immunomic analysis of the repertoire of T-cell specificities for influenza A virus in humans. J. Virol. 2008; 82:12241-12251.
30. Bui H H, Peters B, Assarsson E, Mbawuike I, Sette A. Ab and T cell epitopes of influenza A virus, knowledge and opportunities. Proc Natl Acad Sci USA. 2007; 104:246-251.
31. Gianfrani C, Oseroff C, Sidney J, Chesnut R W, Sette A. Human memory CTL response specific for influenza A virus is broad and multispecific. Hum Immunol. 2000; 61:438-452.
32. Sercarz E E, Lehmann P V, Ametani A, Benichou G, Miller A, Moudgil K. Dominance and crypticity of T cell antigenic determinants. Annu Rev Immunol. 1993; 11:729-766.
33. Oelke M, Maus M V, Didiano D, June C H, Mackensen A, Schneck J P. Ex vivo induction and expansion of antigen-specific cytotoxic T cells by HLA-Ig-coated artificial antigen-presenting cells. Nat. Med. 2003; 9:619-624.
34. Oelke M, Moehrle U, Chen J L, et al. Generation and purification of CD8+ melan-A-specific cytotoxic T lymphocytes for adoptive transfer in tumor immunotherapy. Clin Cancer Res. 2000; 6:1997-2005.
35. Slansky J E, Rattis F M, Boyd L F, et al. Enhanced antigen-specific antitumor immunity with altered peptide ligands that stabilize the MHC-peptide-TCR complex. Immunity. 2000; 13:529-538.
36. Lehner P J, Wang E C, Moss P A, et al. Human HLA-A0201-restricted cytotoxic T lymphocyte recognition of influenza A is dominated by T cells bearing the V beta 17 gene segment. J Exp Med. 1995; 181:79-91.
37. Moss P A, Moots R J, Rosenberg W M, et al. Extensive conservation of alpha and beta chains of the human T-cell antigen receptor recognizing HLA-A2 and influenza A matrix peptide. Proc Natl Acad Sci USA. 1991; 88:8987-8990.
38. Deng Y, Jing Y, Campbell A E, Gravenstein S. Age-related impaired type 1 T cell responses to influenza: reduced activation ex vivo, decreased expansion in CTL culture in vitro, and blunted response to influenza vaccination in vivo in the elderly. J. Immunol. 2004; 172:3437-3446.
39. Yager E J, Ahmed M, Lanzer K, Randall T D, Woodland D L, Blackman M A. Age-associated decline in T cell repertoire diversity leads to holes in the repertoire and impaired immunity to influenza virus. J Exp Med. 2008; 205:711-723.
40. Fagnoni F F, Vescovini R, Passeri G, et al. Shortage of circulating naive CD8(+) T cells provides new insights on immunodeficiency in aging. Blood. 2000; 95:2860-2868.
41. Almanzar G, Schwaiger S, Jenewein B, et al. Long-term cytomegalovirus infection leads to significant changes in the composition of the CD8+ T-cell repertoire, which may be the basis for an imbalance in the cytokine production profile in elderly persons. J. Virol. 2005; 79:3675-3683.
42. Khan N, Shariff N, Cobbold M, et al. Cytomegalovirus seropositivity drives the CD8 T cell repertoire toward greater clonality in healthy elderly individuals. J. Immunol. 2002; 169:1984-1992.
43. Posnett D N, Sinha R, Kabak S, Russo C. Clonal populations of T cells in normal elderly humans: the T cell equivalent to “benign monoclonal gammapathy”. J Exp Med. 1994; 179:609-618.
44. Kim J H, Skountzou I, Compans R, Jacob J. Original antigenic sin responses to influenza viruses. J. Immunol. 2009; 183:3294-3301.
45. Davenport F M, Hennessy A V, Francis T, Jr. Epidemiologic and immunologic significance of age distribution of antibody to antigenic variants of influenza virus. J Exp Med. 1953; 98:641-656.
46. Welsh R M, Selin L K. No one is naive: the significance of heterologous T-cell immunity. Nat Rev Immunol. 2002; 2:417-426.
47. Chen H D, Fraire A E, Joris I, Brehm M A, Welsh R M, Selin L K. Memory CD8+ T cells in heterologous antiviral immunity and immunopathology in the lung. Nat. Immunol. 2001; 2:1067-1076.
48. McElhaney J E, Xie D, Hager W D, et al. T cell responses are better correlates of vaccine protection in the elderly. J. Immunol. 2006; 176:6333-6339.
49. Man S, Newberg M H, Crotzer V L, et al. Definition of a human T cell epitope from influenza A non-structural protein 1 using HLA-A2.1 transgenic mice. Int Immunol. 1995; 7:597-605.
50. Kasprowicz V, Ward S M, Turner A, et al. Defining the directionality and quality of influenza virus-specific CD8+ T cell cross-reactivity in individuals infected with hepatitis C virus. J Clin Invest. 2008; 118:1143-1153.
51. Fagnoni F F, Vescovini R, Passeri G, Bologna G, Pedrazzoni M, Lavagetto G, Casti A, Franceschi C, Passeri M, Sansoni P: Shortage of circulating naive CD8(+) T cells provides new insights on immunodeficiency in aging. Blood 2000, 95:2860-2868.

1. A method of inducing an immune response against an influenza virus, comprising administering to an individual a composition comprising two or more purified peptides selected from the group consisting of NMLSTVLGV (SEQ ID NO:2), CVNGSCFTV (SEQ ID NO:3), SLENFRAYV (SEQ ID NO:4), IMDKNIILKA (SEQ ID NO:5), SLCPIRGWAI (SEQ ID NO:6), and FMYSDFHFI (SEQ ID NO:7).

2. The method of claim 1 wherein the individual is at least 65 years old.

3. The method of claim 1 wherein the composition comprises two purified peptides selected from the group.

4. The method of claim 1 wherein the composition comprises three purified peptides selected from the group.

5. The method of claim 1 wherein the composition comprises four purified peptides selected from the group.

6. The method of claim 1 wherein the composition comprises five purified peptides selected from the group.

7. The method of claim 1 wherein the composition comprises six purified peptides.

8. The method of claim 1 wherein the elderly individual is at least 67 years old.

9. The method of claim 1, wherein the administering comprises injection.

10. The method of claim 1, wherein the injection is subcutaneous or intramuscular.

11. The method of claim 1, wherein the administering comprises inhalation.

12. The method of claim 11, wherein the inhalation comprises inhaling a nasal aerosol or mist.

13. The method of claim 1, wherein the composition further comprises an adjuvant.
